(19) United States
(12) Patent Application Publication
(10) Pub. No.: US 2017/0337350 A1
(43) Pub. Date: Nov. 23, 2017

(54) USER TERMINAL APPARATUS AND METHOD FOR DRIVING USER TERMINAL APPARATUS

(71) Applicant: Samsung Electronics Co., Ltd., Suwon-si, Gyeonggi-do (KR)

(72) Inventors: Sun-kyung KIM, Kyiv (UA); Ievgenii IAKISHYN, Kyiv (UA); Mykola ALIEKSIEIEV, Kyiv (UA); Yurii TSIVUN, Borispil Kyiv (UA)

(21) Appl. No.: 15/533,187

(22) PCT Filed: Nov. 18, 2015
(86) PCT No.: PCT/KR2015/012410

§ 371 Date: Jun. 5, 2017

(30) Foreign Application Priority Data
Dec. 30, 2014 (KR) 10-2014-0193716

(51) Int. Cl. G06F 19/00; A61B 5/00; G06Q 10/10; G06F 3/0488; G06F 3/0482; G06T 19/20; A61B 5/11; G06F 3/0481; G06F 17/28

(52) U.S. Cl. CPC G06F 19/363; A61B 5/1118; A61B 5/7425; A61B 5/744; G06Q 10/1093; G06F 3/04815; G06F 19/322; A61B 5/0015; G06F 3/04886; G06F 3/0482; G06T 19/20; G06F 19/3418; G06T 2200/24; G06T 2219/2012; G06F 17/289

(57) ABSTRACT

A user terminal apparatus, a method for driving the user terminal apparatus, and a computer readable recording medium are provided. The user terminal apparatus includes a display configured to display a user model personalized to a user and to recommend a plurality of graphic images associated with a physical condition of a user through one zone of the displayed user model, Cand an information visualization processor configured to, in response to one image being selected from the plurality of recommended graphic images, control the display to apply the selected graphic image to the user model.

### TECHNICAL FIELD

Apparatuses and methods consistent with the present invention relate to a user terminal apparatus and a method for driving the user terminal apparatus, and more particularly, to a user terminal apparatus and a method for driving the user terminal apparatus, for easily checking a physical condition of a user based on a personalized user model in, for example, a healthcare system, Web, wearable technology, an educational system of students for a gaming system and medical generalization, and various technologies for understanding of physical conditions of other people.

### BACKGROUND ART

Recently, there has been an attempt to use the ‘3D hologram avatar’ in clinical trials of patients, in accordance with current trends. The 3D hologram avatar has been developed to enhance the stability and accuracy of medical treatment and is capable of being personalized according to a body condition of a patient. By virtue of the 3D hologram avatar, body conditions of an actual human body, such as a pulse and a blood pressure as well as an age, a body shape, and a weight, are capable of being precisely embodied so as to enable bedside and clinical training of predicting symptom and reaction of patients.

### DISCLOSURE OF INVENTION

### Technical Problem

However, although such a typical 3D avatar precisely embodies a body condition of an actual human body, this is not friendly to a user in that the 3D avatar is excessively limited only to display of a personalized 3D image of a human body, a user such as a patient is capable of freely seeing the 3D image anytime and anywhere, and an operation of recording a physical condition is limited.

### Solution to Problem

Exemplary embodiments of the present invention overcome the above disadvantages and other disadvantages not described above. Also, the present invention is not required to overcome the disadvantages described above, and an exemplary embodiment of the present invention may not overcome any of the problems described above.

The present invention provides a user terminal apparatus and a method for driving the user terminal apparatus, for easily checking a physical condition of a user based on a personalized user model in, for example, a healthcare system, Web, wearable technology, an educational system of students for a gaming system and medical generalization, and various technologies for understanding of physical conditions of other people.

According to an aspect of the present invention, a user terminal apparatus includes a display configured to display a user model personalized to a user and to recommend a plurality of graphic images associated with a physical condition of a user through one zone of the displayed user model, and an information visualization processor configured to, in response to one image being selected from the plurality of recommended graphic images, control the display to apply the selected graphic image to the user model.

The user terminal apparatus may further include a storage configured to store medical information, and a sensor configured to acquire data associated with the physical condition, wherein the information visualization processor recommend the plurality of graphic images based on at least one of the stored medical information and the acquired data.

The sensor may include at least one sensor for detection of a physical activity level of the user.

The user terminal apparatus may further include a communication interface operatively associated with a wearable device that the user wears in order to measure the physical condition of the user, and the information visualization processor may acquire data associated with the physical condition through the communication interface.

The information visualization processor may display different types of symptom associated with the physical condition as the plurality of graphic images.

The display may further display a calendar showing the physical condition of the user according to date change, and in response to a date being selected from the calendar, may further display a graphic image of the selected date.

The information visualization processor may change information associated with the physical condition into a language selected by the user and display the information on the display in order to overcome a language issue.

According to an aspect of the present invention, a method for driving a user terminal apparatus includes displaying a user model personalized to a user and recommending a plurality of graphic images associated with a physical condition of a user through one zone of the displayed user model, by a display, and in response to one image being selected from the plurality of recommended graphic images, controlling the display to apply the selected graphic image to the user model.

The method may further include storing medical information, and acquiring data associated with the physical condition, wherein the controlling may include recommending the plurality of graphic images based on at least one of the stored medical information and the acquired data.

The acquiring of the data may include acquiring the data using at least one sensor for detection of a physical activity level of the user.

The method may further included being operatively associating a wearable device that the user wears in order to measure the physical condition of the user, wherein the acquiring may include acquiring data associated with the physical condition provided by the wearable device.

The controlling may include displaying different types of symptom associated with the physical condition as the plurality of graphic images.

The displaying may include further displaying a calendar showing the physical condition of the user according to date change, and in response to a date being selected from the calendar, further displaying a graphic image of the selected date.

The displaying may include changing information associated with the physical condition into a language selected by the user and displaying the information on the display in order to overcome a language issue.

According to the diverse exemplary embodiments of the present invention, a physical condition may be visually displayed in the form of a graphic image so as to simply check symptom without a separate typing procedure, which may be friendly to a user and may be performed in real time.

Additional and/or other aspects and advantages of the invention will be set forth in part in the description which follows and, in part, will be obvious from the description, or may be learned by practice of the invention.

### ADVANTAGEOUS EFFECTS OF INVENTION

Like in an exemplary embodiment of the present invention, a physical condition may be visually displayed in the form of a graphic image such that the user may check symptom via simple click without separate typing process. This may be friendly to a user and may be performed in real time. In addition, an issue in terms of verbal communication between a patient and a doctor may be overcome, and the type or intensity of symptom may be accurately estimated through prediction based on medical information.

### BRIEF DESCRIPTION OF DRAWINGS

The above and/or other aspects of the present invention will be more apparent by describing certain exemplary embodiments of the present invention with reference to the accompanying drawings, in which:

FIG. 1 is a diagram illustrating an electronic health record (HER) system according to an exemplary embodiment of the present invention,

FIG. 2 is a diagram illustrating symptom indicated by click of a personalized user model according to an exemplary embodiment of the present invention,

FIG. 3 is a block diagram illustrating a detailed structure of a user terminal apparatus 1 or a user terminal apparatus 2 of FIG. 1,

FIG. 4 is a block diagram illustrating another detailed structure of the user terminal apparatus 1 or the user terminal apparatus 2 of FIG. 1,

FIG. 5 is a flowchart illustrating a driving procedure of the user terminal apparatus 1 and the user terminal apparatus 2 of FIG. 1,

FIG. 6 is a flowchart illustrating a driving procedure of the user terminal apparatus 1 of FIG. 1,

FIG. 7 is a flowchart illustrating a driving procedure of the user terminal apparatus 2 of FIG. 1,

FIGS. 8 to 12 are diagrams for explanation of a procedure for generating and checking a graphic image associated with a physical condition of a user,

FIG. 13 is a diagram illustrating an example of a user interface (UI) according to an exemplary embodiment of the present invention,

FIG. 14 is a diagram illustrating an image obtained by dividing a head part illustrated in FIG. 13(a) into detailed zones,

FIG. 15 is a diagram illustrating a procedure for additionally recording symptom,

FIG. 16 is a diagram illustrating the case in which selected symptom is displayed in a user model when a user selects the symptom,

FIG. 17 is a diagram illustrating an example of fly out animation,

FIG. 18 is a diagram illustrating an example of an image when a calendar item of an image is selected, and

FIG. 19 is a diagram illustrating an example of an image of FIG. 18(b).

### BEST MODE FOR CARRYING OUT THE INVENTION

### Mode for the Invention

Certain exemplary embodiments of the present invention will now be described in greater detail with reference to the accompanying drawings.

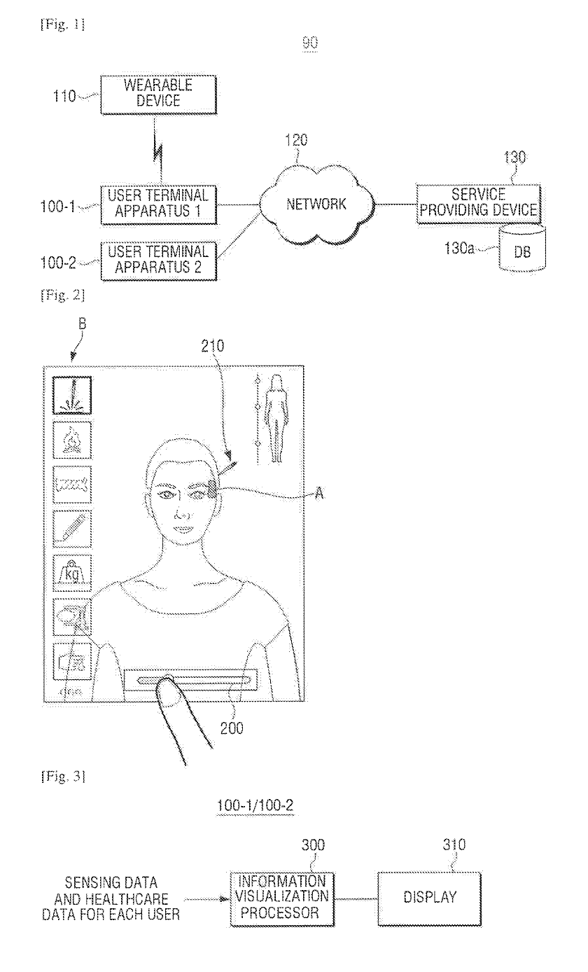

FIG. 1 is a diagram illustrating an electronic health record (HER) system according to an exemplary embodiment of the present invention.

As illustrated in FIG. 1, the HER system 90 according to the embodiment of the present invention may include all or some of a user terminal apparatus 1 100-1, a user terminal apparatus 2 100-2, a wearable device 110, a communication network 120, and a service providing device 130.

Here, inclusion of all or some means that the user terminal apparatus 1 100-1 or the user terminal apparatus 2 100-2 may be omitted and the wearable device 110 may also be omitted, but in the specification, the HER system 90 includes all the components for a sufficient understanding of the present invention.

The user terminal apparatus 1 100-1 may include an image display apparatus including a mobile terminal apparatus such as a smart phone, an MP3 player, a plasma display panel (PDP), and a notebook computer, and a fixed terminal apparatus positioned at a fixed place, such as a desk top computer and a television (TV). The user terminal apparatus 1 100-1 may be, for example, a terminal apparatus of a patient side and may use a medical service provided by the service providing device 130.

For example, the user terminal apparatus 1 100-1 may execute an application (or a tool-kit) stored therein in order to use the medical service provided by the service providing device 130. As such, a user may generate a user profile and determine a user model personalized to the user, for example, a 3D avatar based on the generated user profile and acquired data associated with physical condition of the user. Here, the user model may be obtained by displaying pain (or symptom) based on medical information (or medical knowledge) or pain at connection parts of a body as a graphic image. Here, a pain type may be visually differently displayed and pain intensity may be further visually displayed using the graphic image.

In more detail, the user terminal apparatus 1 100-1 may acquire data associated with the physical condition of the user detected by sensors and may automatically add the data to the user profile. For example, the user terminal apparatus 1 100-1 may detect a physical activity level of the user using sensors installed in the user terminal apparatus 1 100-1, such as a gyroscope and an acceleration sensor, and may add the detected data to the profile. In addition, the user terminal apparatus 1 100-1 may communicate with the wearable device 110 that the user wears to acquire data associated with the physical condition of the user and add the data to the profile. Here, the wearable device 110 may include a device such as bracelet, glasses, and a watch, may include different specific medical devices for detection of pulse, body temperature, and heartbeat data as an external measurement device, and may synchronize with the user terminal apparatus 1 100-1.

The user terminal apparatus 1 100-1 may receive the user emotion data that is manually input by the user. The user terminal apparatus 1 100-1 may propose (or recommend) intuitive graphic images with different shapes or differently predicted symptoms based on data that is input by the user or is previously collected, to the user and may receive selection from the user. The intuitive graphic images may include a graphic image for estimation of symptom of connected zones in more detail. Accordingly, the user terminal apparatus 1 100-1 may display symptoms selected on the personalized user model so as to allow the user to view the symptoms.

Likewise, the user terminal apparatus 1 100-1 may compile statistics, for example, everyday in consideration of automatically detected data and data that is manually input by the user and may visualize the physical condition of the user based on the statistics. Here, visualization may refer to an operation for allowing the user to visually view at least one of the type and intensity of symptom on the personalized user model.

In addition, the user terminal apparatus 1 100-1 may display a calendar associated with a physical condition on a screen image according to user request. For example, in response to a specific date being selected from the calendar by the user, the user terminal apparatus 1 100-1 may visually display a physical condition determined at the corresponding date. In other words, a user model and a graphic image indicated on the user model may be displayed on the screen image together. As such, the user may easily check pain or symptom for each day, month, and year and may manage a symptom history according to a time change.

In addition, the user terminal apparatus 1 100-1 may change information (or data) about the physical condition input by the user to a plurality of languages. For example, since there is difficulties in verbal communication between a patient and a doctor or a patient and a consultant, the user terminal apparatus 1 100-1 according to the exemplary embodiment of the present invention may translate the collected information associated with the physical condition of the user into a plurality of languages during transmission of the associated information to the service providing device 130.

Needless to say, the translation may be performed by the service providing device 130, but entire data of the user terminal apparatus 1 100-1 may be stored in the user terminal apparatus 1 100-1 rather than being provided to the service providing device 130, and thus the translation may be performed by the user terminal apparatus 1 100-1. However, a subject that performs the translation is not particularly limited in the exemplary embodiment of the present invention.

The user terminal apparatus 2 100-2 may not be largely different from the user terminal apparatus 1 100-1. However, the user terminal apparatus 2 100-2 may correspond to a terminal apparatus processed by a medical consultant or doctor. Accordingly, the user terminal apparatus 2 100-2 may check image data associated with the physical condition provided by the user of the user terminal apparatus 1 100-1 and may request additional information associated with the physical condition or may further perform an operation such as diagnosis and appointment.

In addition, the result obtained by processing this operation may also be translated into a plurality of languages and stored in the user terminal apparatus 2 100-2 or may be provided to the service providing device 130. Accordingly, the user terminal apparatus 1 100-1 may check the diagnosis result of the physical condition of the user.

As described above, needless to say, the wearable device 110 may include a bracelet or a ring that the user of the user terminal apparatus 1 100-1 wears and a wearable computer such as the Galaxy Gear. The wearable device 110 may include a measurement device such as a thermometer or a pulse meter, for detection of the physical condition of the user. In addition, the wearable device 110 may include a communication module for communication with the user terminal apparatus 1 100-1 and a control module.

The communication network 120 may include any wired and wireless communication networks. Here, the wired communication network may include the Internet such as a cable network or a public switched telephone network (PSTN) and the wireless communication network may include CDMA, WCDMA, GSM, evolved packet core (EPC), long term evolution (LTE), a Wibro network, etc. Needless to say, the communication network 120 according to the exemplary embodiment of the present invention is not limited thereto and may be used in, for example, a cloud computing network in a cloud computing environment as an access network of an advanced next generation mobile communication system. For example, when the communication network 120 is a wired communication network, an access point in the communication network 120 may access a switch center of a telephone office, but the communication network 120 is a wireless communication network, the access point may access SGSN or a gateway GPRS support node (GGSN) managed by a communication company and process data or may access various relays such as base station transmission (BTS), NodeB, and e-NodeB and process data.

The communication network 120 may include an access point. The access point may include a small base station such as a femto or pico base station, which is largely installed in a building. Here, according to classification of a small station, the femto and pico base station may be differentiated according to a maximum number of user terminal apparatuses which access a base station. Needless to say, an access point may include a short-distance communication module for short-distance communication such as ZigBee and Wi-Fi with a user terminal apparatus. The access point may use TCP/IP or a real-time streaming protocol (RTSP) for wireless communication. Here, the short-distance communication may be performed with various standards of ultra wide band communication (UWB) and radio frequency (RF) such as Bluetooth, Zigbee, infrared (IrDA), ultra high frequency (UHF), and very high frequency (VHF) as well as WiFi. Accordingly, the access point may extract a position of a data packet, determine an optimum communication path with respect to the extracted position, and transmit the data packet to a next apparatus, for example, a user terminal apparatus along the determined communication path. The access point may share various circuits in a general network environment and may include, for example, a router, a repeater, a relay, and so on.

The service providing device 130 may be a server managed by a hospital but may be a server managed by a third party such as a consultant. The service providing device 130 may receive and may collectively manage various information items, for example, physical condition information provided by the user terminal apparatus 1 100-1 of a patient side and diagnosis and appointment information provided by the user terminal apparatus 2 100-2 of a doctor side. In addition, the service providing device 130 may be operatively associated with the user terminal apparatus 1 100-1 and the user terminal apparatus 2 100-2 to display the physical condition of the user as the personalized user model and a graphic image of the type, intensity, and so on of symptom, which is indicated on the user model. In reality, all image data items associated with the physical condition of the user may be provided by the service providing device 130, and the user terminal apparatus 1 100-1 and the user terminal apparatus 2 100-2 may execute an application for simply using a service and display an image on a screen image according to a predetermined rule.

The service providing device 130 may include a database (DB) 130a. The DB 130a may store and manage various data items associated with the physical condition of the user. In this case, the various data items associated with the physical condition may be stored in the form of image data of a user model personalized for each user and a graphic image associated with the physical condition, to be inserted into the user model.

FIG. 2 is a diagram illustrating symptom indicated by click of a personalized user model according to an exemplary embodiment of the present invention.

As illustrated in FIG. 2, a user of, for example, the user terminal apparatus 1 100-1 illustrated in FIG. 1 may view detailed symptoms on the personalized user model via one click. Although described in detail later, the user of the user terminal apparatus 1 100-1 may select a menu icon displayed on a background image and execute an application to first display a user model personalized for each user on a screen image. In this case, the user model may be obtained by dividing a body part into three zones of a head part, a torso part, and a leg part and displayed such that the user selects each zone (or region).

In addition, when the user selects a specific zone with a physical condition that the user wants to check, the physical condition of the corresponding zone may be displayed as a graphic image. FIG. 2 illustrates a procedure of inputting symptom of the head part by the user. The inputting procedure may be changed in various ways, but as a representative example, the inputting procedure may be performed such that a graphic image is displayed on a touched zone when the user touches the specific zone with a finger or a selectable zone is recommended to allow the user to select the zone. The former case is described with reference to FIG. 2 but the latter case will be described later. For example, when the user touches a specific zone A as illustrated in FIG. 2, the user terminal apparatus 1 100-1 may allow the user to input intensity of pain. To this end, as illustrated in FIG. 2, a control lever 200 may be displayed on a screen image and a graphic image 210 may be displayed in a corresponding zone according to a control level.

Images with various shapes indicating intensity of pain may be set as an example of a graphic image in one zone B of the screen image. The image may be one image that is selected by the user from a plurality of images according to a pain degree adjusted through the control lever 200 and the corresponding selected image may be activated to exhibit different color from other images. Needless to say, the image may be displayed in various forms as long as the image may be visually identified. For example, the image may be changed in various ways, for example, of being highlighted or changing a shape of an edge line.

Information of the physical condition of the user, input via the procedure, may be stored together with a date, and an image of a physical condition, input according to a user request, that is, a user model and a graphic image associated with symptom on the user model may be displayed on a screen image. The image may be provided in the same form irrespective of patient request or doctor request.

FIG. 3 is a block diagram illustrating a detailed structure of the user terminal apparatus 1 100-1 or the user terminal apparatus 2 100-2 of FIG. 1.

For convenience of description, with reference to FIG. 3 together with the user terminal apparatus 1 100-1 of FIG. 1, the user terminal apparatus 1 100-1 illustrated in FIG. 1 may include all or some of an information visualization processor 300 and a display 310.

Here, inclusion of all or some means that some components such as the information visualization processor 300 or the display 310 are omitted or some components such as the information visualization processor 300 is integrated with another component such as the display 310, and in the specification, the user terminal apparatus 1 100-1 includes all the components for a sufficient understanding of the present invention. For example, the information visualization processor 300 may form an independent device and the display 310 may be structurally separated from the independent device, and thus some components may be omitted.

In response to user information being input, the information visualization processor 300 may determine a user model identified according to a user height, weight, or the like, for example, a 3D avatar. During this procedure, the 3D avatar may be determined according to user selection from candidate avatars proposed on a screen image.

The information visualization processor 300 may collect data associated with the physical condition of the user through various paths. For example, a physical activity level may be detected through sensors included in the user terminal apparatus 1 100-1, and data associated with a body temperature, a pulse, or the like of the user may be acquired from an external device such as the aforementioned wearable device 110. In addition, various types of graphic images predictable based on medical information stored in the user terminal apparatus 1 100-1, for example, symptoms may be proposed such that the user selects symptom.

As such, in response to data associated with symptom being collected, the information visualization processor 300 may insert a graphic image associated with the type or shape of pain into a user model and provide the user model to the display 310. For example, the information visualization processor 300 may store information associated with symptom in the service providing device 130 of FIG. 1, receive corresponding information, and then perform image processing.

During this process, according to separate user request, the information visualization processor 300 may translate information into a specific language selected by the user and provide the information.

The entire collected data associated with the physical condition of the user may be provided to the service providing device 130 of FIG. 1, the service providing device 130 may generate a user model into which graphic is inserted, and the information visualization processor 300 may receive the generated image data from the service providing device 130 and process the image data, and thus the exemplary embodiment of the present invention may not be particularly limited to the above description.

In the case of the user terminal apparatus 2 100-2 used by a medical consultant or a doctor, the information visualization processor 300 may check the physical condition of the user and request additional information or may perform an operation such as diagnosis or appointment. Sufficient description has been given above in this regard and thus more description will not be given here.

The information visualization processor 300 may operate in the form of one piece of software. In other words, both a controlling operation and an information generating operation for information visualization may be processed by one program. Needless to say, the information visualization processor 300 may be configured to include a central processing unit (CPU) and a memory. The memory may include a program for generating information and execute the program according to control of the CPU. However, needless to say, a specific module of the program may be generated in terms of hardware, and thus the exemplary embodiment of the present invention may not be particularly limited to the form of the program.

The display 310 may display a user model personalized for each user and a graphic image associated with the physical condition of the user in the user model according to control of the information visualization processor 300. The display 310 may include a touch panel so as to input an inputting procedure through an interface with the user using the touch panel. For example, the display 310 may recommend various forms of graphic images predictable with respect to specific symptom associated with the physical condition of the user to the user so as to allow the user to select a graphic image among the graphic images. Then, the user may select one graphic image through a screen touch operation.

In addition, when the user requests a calendar with respect to the physical condition on the screen image, the display 310 may display the calendar, and when the user selects a specific date in the calendar, the display 310 may additionally display a physical condition, that is, symptom of the selected date in the form of a user model and a graphic image, which will be described below in detail.

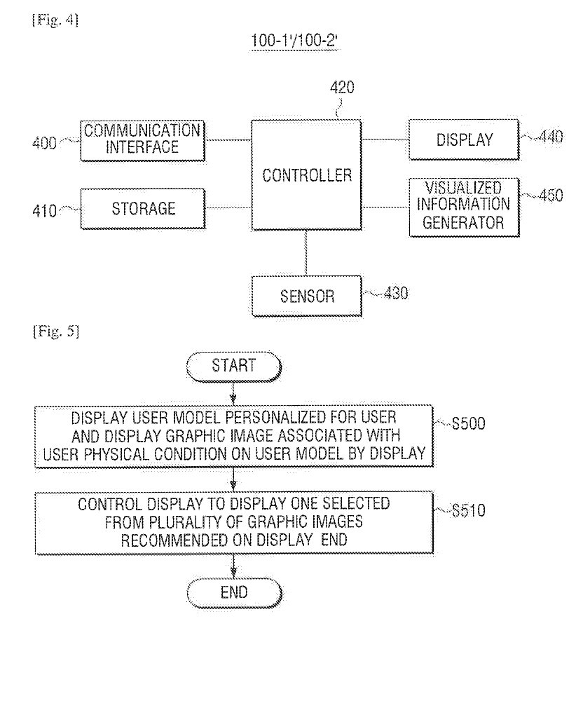

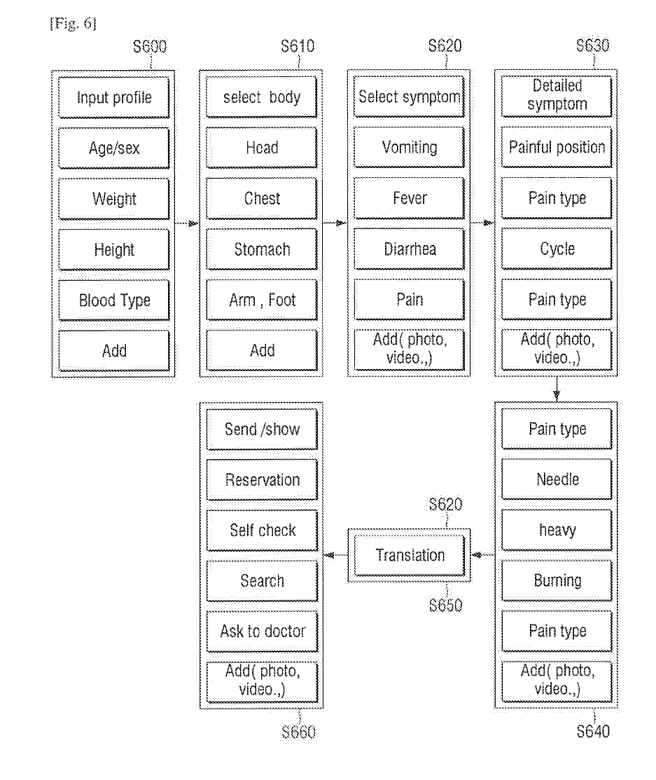

FIG. 4 is a block diagram illustrating another detailed structure of the user terminal apparatus 1 100-1 or the user terminal apparatus 2 100-2 of FIG. 1.

For convenience of description, with reference to FIG. 4 together with the user terminal apparatus 1 100-1 of FIG. 1, the user terminal apparatus 1 100-1 illustrated in FIG. 1 according to an exemplary embodiment of the present invention may include all or some of a communication interface 400, a storage 410, a controller 420, a sensor 430, the display 440, and the visualized information generator 450.

Here, inclusion of all or some means that some components such as the storage 410 are omitted or the visualized information generator 450 is integrated with another component such as the controller 420, and in the specification, the user terminal apparatus 1 100-1 includes all the components for a sufficient understanding of the present invention.

The communication interface 400 may communicate with the wearable device 110 or communicate with the service providing device 130 through the communication network 120. The communication interface 400 may perform direct communication with the wearable device 110. For example, the communication interface 400 may acquire data associated with the physical condition of the user who processes the user terminal apparatus 1 100-1 and transmit the data to the controller 420 via communication with the wearable device 110. For example, the communication interface 400 may receive body temperature information, pulse information, and so on from the wearable device 110.

In addition, the communication interface 400 may download an application for using a service according to an exemplary embodiment of the present invention through communication with the service providing device 130 and store the application in the storage 410 or the visualized information generator 450, may receive image data associated with the physical condition of the user, that is, a user model and a symptom related graphic image displayed in the user model, may image-process the image data, and may transmit the image data to the controller 420 so as to display the image data on the display 440.

The storage 410 may temporally store various information items processed by the user terminal apparatus 1 100-1. For example, when decoding is performed through the communication interface 400, the store 410 may temporally store the decoding result. In addition, the storage 410 may store an application for using a service.

The controller 420 may control an overall operation of the communication interface 400, the storage 410, the sensor 430, a display 440, a visualized information generator 450, and so on which constitute the user terminal apparatus 1 100-1. For example, when a user selects a menu icon displayed on the display 440 in order to use a service, the controller 420 may execute the application stored in the storage 410 to access the service providing device 130 and receive various information items processed according to user input. For example, when the user forms a graphic image about information associated with the user physical condition, that is, symptom, the controller 420 may receive a list of the graphic image and provide the list to the visualized information generator 450. Accordingly, the controller 420 may receive visualized information generated by the visualized information generator 450 and display the visualized information on the display 440.

In more detail, the controller 420 may perform an operation for displaying a symptom type (or shape) or symptom intensity as a graphic image in a user model personalized for the user using information input by the user with respect to the user physical condition or physical condition related data that is automatically acquired through the sensor 430. In addition, the controller 420 may show a pre-generated user model and a graphic image according to user request. Except for this, the controller 420 is not largely different from the aforementioned information visualization processor 300, and thus a description of the controller 420 may be substituted with the above description of the information visualization processor 300. In reality, the controller 420 and the visualized information generator 450 may be integrated with each other to constitute the information visualization processor 300.

The controller 420 according to the exemplary embodiment of the present invention may include a CPU and a memory. When the user terminal apparatus 1 100-1 begins to operate, the CPU may call a program stored in the visualized information generator 450, store the program in the memory, and then execute the program. Alternatively, the CPU may control the visualized information generator 450 to execute the internal program and to receive the processing result. In this case, the processing result may be image data obtained by inserting a graphic image into the user model.

The sensor 430 may include a gyroscope and an acceleration sensor. The sensors may be used to detect a user physical activity level. When the user moves the sensors, the sensors may detect data about the movement and provide the data to the controller 420. In addition, the controller 420 may provide related data to the visualized information generator 450.

The display 440 is not largely different from the display 310 of FIG. 3, and thus a description of the display 440 may be substituted with the above description of the display 310.

The visualized information generator 450 may perform the same or similar operation to the information visualization processor 300 of FIG. 3. For example, the visualized information generator 450 may receive data associated with the physical condition of the user, that is, user symptom provided by the service providing device 130 in the form of a list and may insert a graphic image into a user model based on the list information. To this end, the visualized information generator 450 may store a program for the above operation and execute the stored program according to control of the controller 420. Needless to say, some modules of the program may be generated in terms of hardware, and thus the exemplary embodiment of the present invention may not be particularly limited to the configuration. Other description of the visualized information generator 450 is not largely different from the information visualization processor 300 of FIG. 3, and thus a description of the visualized information generator 450 may be substituted with the above description of the information visualization processor 300.

FIG. 5 is a flowchart illustrating a driving procedure of the user terminal apparatus 1 100-1 and the user terminal apparatus 2 100-2 of FIG. 1.

For convenience of description, with reference to FIG. 5 together with the user terminal apparatus 1 100-1 of FIG. 1, the user terminal apparatus 1 100-1 according to an exemplary embodiment of the present invention may display a user model personalized for each user on a screen image of a display and display a physical condition of a user as a graphic image on the user model (S500).

To this end, the user terminal apparatus 1 100-1 may previously perform an operation for inputting data for displaying a graphic image of the user physical condition on the personalized user model. For example, a graphic image may be generated based on data about a user physical activity level acquired using internal sensors or predictable forms of symptom may be proposed and a graphic image may be generated according to user selection. In addition, the predictable forms of graphic images may be generated with further reference to internal medical information.

Various information items associated with the graphic image inserted into the user mode may be displayed with various languages, which may be useful to overcome an issue in terms of verbal communication between a patient and a doctor.

The user terminal apparatus 1 100-1 may control a display to insert a graphic image into a user model based on data associated with a physical condition and to display the user model. That is, the user terminal apparatus 1 100-1 may insert the graphic image into the user model to generate image data and provide the image data.

For example, the user terminal apparatus 1 100-1 may control the display to display one selected from a plurality of recommended graphic images and control the display to insert the corresponding selected graphic image and display the user model on the display.

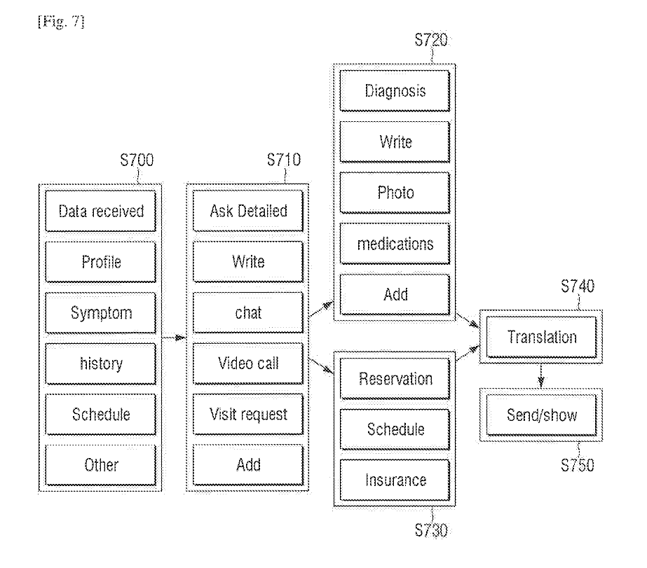

FIG. 6 is a flowchart illustrating a driving procedure of the user terminal apparatus 1 100-1 of FIG. 1.

Referring to FIG. 6 together with FIG. 1, the user terminal apparatus 1 100-1 according to an exemplary embodiment of the present invention may access a server and input user information to generate a user profile (S600). To this end, the user may input various information items such as an age, a sex, a height, a weight, and a blood type. Accordingly, a user model personalized for the user may be determined. During this process, the user terminal apparatus 1 100-1 may propose candidate user models such that the user selects a user model.

In addition, the user may select a body part in detail (S610). For example, the body may be divided in detail into a head part, a torso part, and an arm and leg part such that the user selects the body part.

In response to a specific part being selected, the user terminal apparatus 1 100-1 may allow the user to select symptom of the corresponding part (S620). During this process, the user may add a picture, related video, or the like.

In response to symptom being selected, the type (or shape), intensity, and so on of the symptom may be input (S630 and S640). For example, in the case of pain, a type of the pain or intensity of the pain may be input or one of graphic images of a recommended candidate group may be selected, which has been already exemplified in FIG. 2.

Data generated over operations 5600 to 5640 may be translated into various languages (S650). Here, the generated data may include related information of symptom and so on. Management in various forms of languages may be useful to overcome an issue in terms of verbal communication between a patient and a doctor.

When this process is completed, the user may store physical condition related data in the user terminal apparatus 1 100-1 or transmit the related data to the service providing device 130, and receive and check the related data anytime as necessary (S660). During this process, the user may perform an operation of an additional visit appointment.

FIG. 7 is a flowchart illustrating a driving procedure of the user terminal apparatus 2 100-2 of FIG. 1.

For convenience of description, with reference to FIG. 7 together with the user terminal apparatus 1 100-1 of FIG. 1, the user terminal apparatus 2 100-2 according to an exemplary embodiment of the present invention may receive patient data according to visit request of a user or may directly access the service providing device 130 and may receive the patient data (S700).

As such, a doctor or a consultant who processes the user terminal apparatus 2 100-2 may check a user model personalized for each user, which is formed based on directly and indirectly input data, and a symptom related graphic image on the user model.

After related symptom is checked through an image, the user terminal apparatus 2 100-2 may request more detailed additional information or materials (S710). Information about the request may be transmitted to the user terminal apparatus 1 100-1 through the service providing device 130.

In addition, the user of the user terminal apparatus 2 100-2 may diagnose a physical condition of the user of the user terminal apparatus 1 100-1 through the additionally provided information or materials and a previous symptom related image (S720) or may determine or adjust schedule through visit appointment (S730).

Then the user terminal apparatus 2 100-2 may form various information items such as additional information or materials with various languages (S740).

In addition, the user of the user terminal apparatus 2 100-2 may store data therein or may provide the data to the service providing device 130 outside the user of the user terminal apparatus 2 100-2 such that the user terminal apparatus 1 100-1 or the user terminal apparatus 2 100-2 checks the data anywhere.

FIGS. 8 to 12 are diagrams for explanation of a procedure for generating and checking a graphic image associated with a physical condition of a user.

For convenience of description, with reference to FIGS. 8 to 11 together with FIGS.

1 and 6, the user terminal apparatus 1 100-1 according to an exemplary embodiment of the present invention may generate a screen image illustrated in FIG. 8 and display the screen image according to information input operation 5600 of FIG. 6. As illustrated in FIG. 8, the user terminal apparatus 1 100-1 may display a user model 800 determined according to a height, a weight, or the like of a user in a central portion of the screen image. In addition, a body part image 810 may be displayed in a first zone A of a left portion such that the user selects a detailed body portion. Detailed user information may be displayed in a second zone B of a right portion. In addition, a calendar item 820 by which a physical condition of the user is viewable in the form of a calendar may be formed in a lower portion of the second zone B. In addition, an image indicating that symptom is inputable in the form of voice.

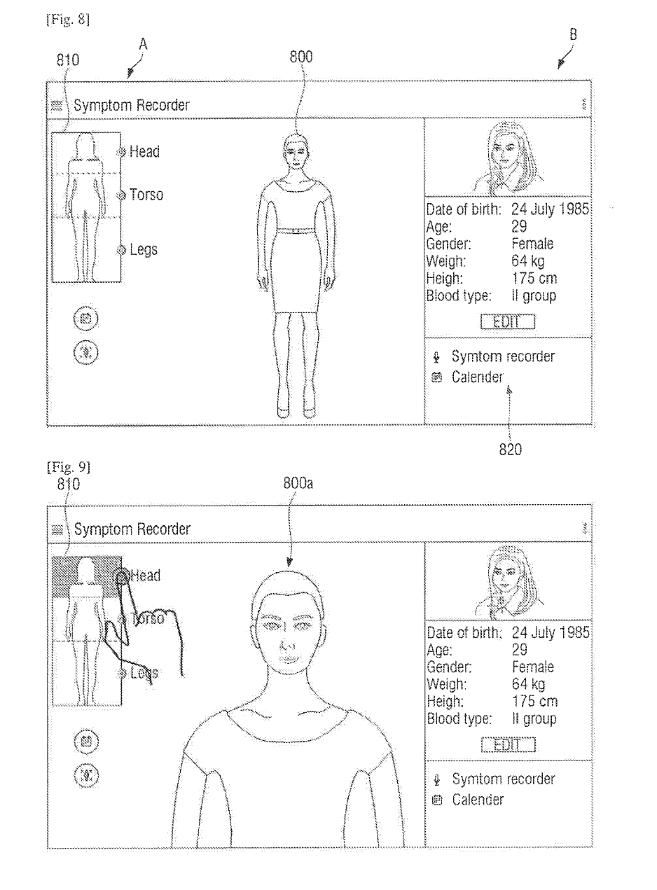

Then in order to input symptom of a detailed body part, the user may select a head part in the body part image 810 of the first zone A as illustrated in FIG. 9. As such, the user terminal apparatus 1 100-1 may enlarge and display a user model 800a in the central portion of a screen such that the user views the user head portion in more detail.

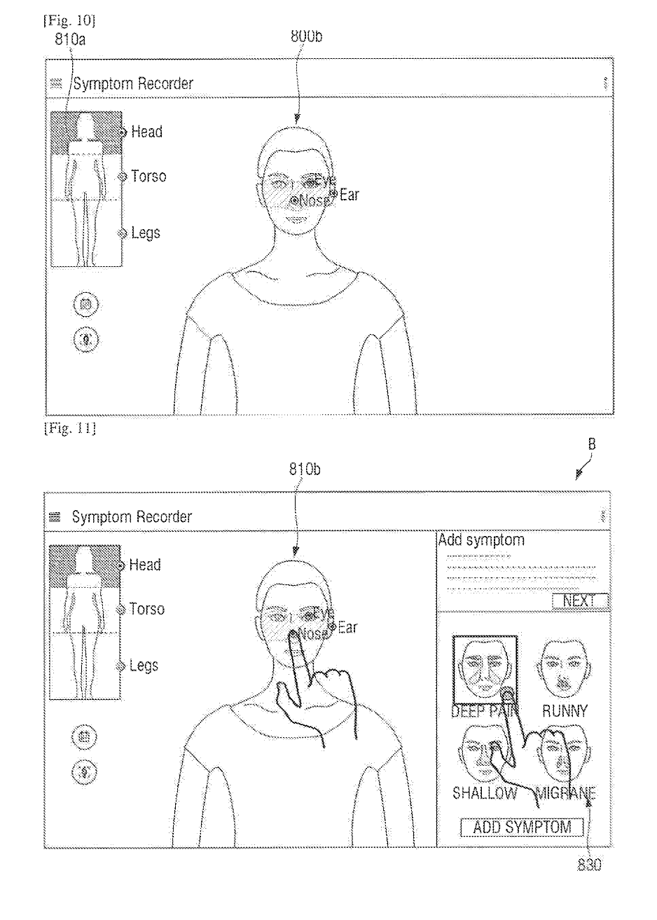

In this case, the user terminal apparatus 1 100-1 may display a user model 800b to which a graphic image associated with a physical condition, for example, symptom is added, as illustrated in FIG. 10, according to data that is automatically pre-collected, that is, data collected via sensing data. In this case, information indicating that the head part is currently displayed may be signaled through a body part image 810a of the first zone A.

In addition, when the user selects a corresponding zone in order to input symptom of a nose part as illustrated in FIG. 11, the user terminal apparatus 1 100-1 may recommend a candidate graphic image 830 in the second zone B from which user information is deleted. Here, the candidate graphic image 830 may be recommended based on pre-stored medical information and may be an image that is predicted and recommended based on sensing data and so on. The candidate graphic image 830 may correspond to symptom associated with a nose part, but a type of pain and intensity of the pain may be displayed as a graphic image.

In addition, when the user selects one image from the recommended candidate graphic image 830 as illustrated in FIG. 11, information of the selected related graphic image 830 may be stored.

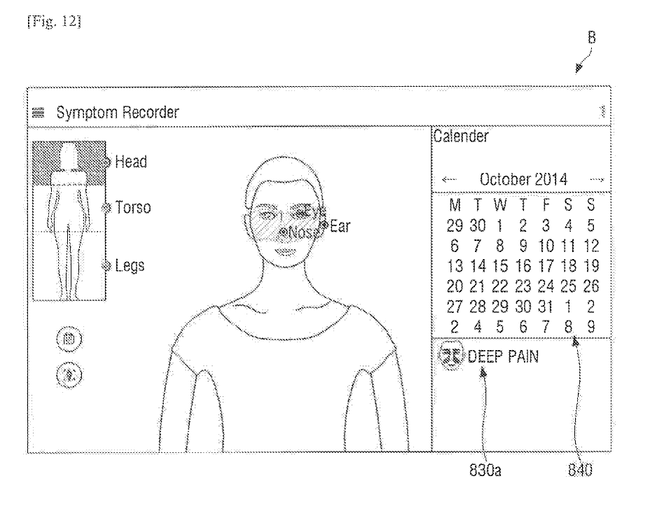

Then when the user selects the calendar item 820 on a screen image of FIG. 8, a screen image illustrated in FIG. 12 may be displayed, and in this case, a calendar 840 may be displayed in the second zone B.

In addition, when the user selects a specific date in the calendar 840 of FIG. 12, a graphic image 830a selected at a specific time point may be reduced and displayed in a lower portion of the calendar 840 as illustrated in FIG. 11, for example.

Needless to say, a method for generating and displaying a graphic image associated with symptom illustrated in FIGS. 8 to 12 may be changed and embodied in various forms. However, like in an exemplary embodiment of the present invention, a physical condition may be visually displayed in the form of a graphic image such that the user may check symptom via simple click without separate typing process. This may be friendly to a user and may be performed in real time. In addition, an issue in terms of verbal communication between a patient and a doctor may be overcome, and the type or intensity of symptom may be accurately estimated through prediction based on medical information.

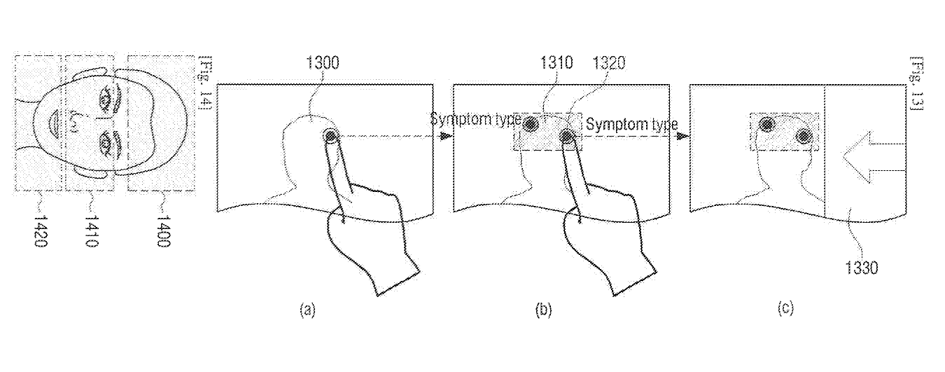

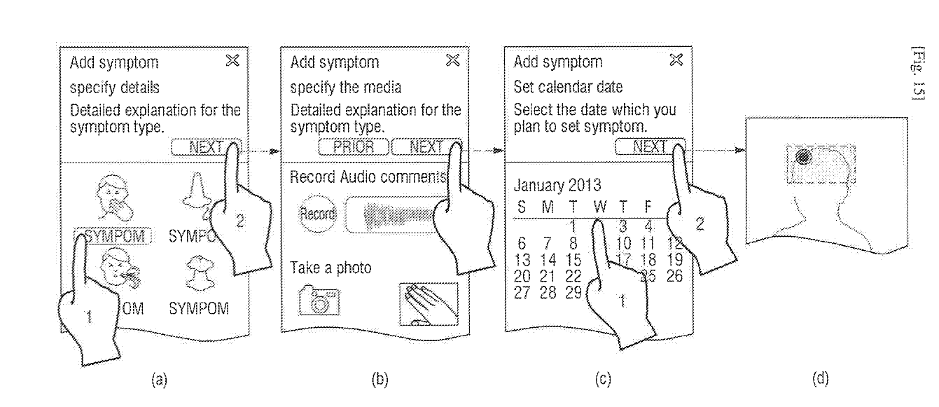

FIG. 13 is a diagram illustrating an example of a user interface (UI) according to an exemplary embodiment of the present invention and FIG. 14 is a diagram illustrating an image obtained by dividing a head part illustrated in FIG. 13(a) into detailed zones.

FIGS. 13(a) to 13(c) may correspond to, for example, portions of images of FIGS. 9 to 11, respectively. For convenience of description, referring to FIGS. 13 and 14 together with FIG. 1, the user terminal apparatus 1 100-1 of FIG. 1 according to an exemplary embodiment of the present invention may display a user model 1300 illustrated in FIG. 13(a) on a screen image according to user request. In this case, the user model 1300 may include a 3D model. Like in the screen image of FIG. 13(a), a user may tap a symptoms zone for recording or checking detailed symptom in the user model 1300. For example, a head part of the user model 1300 may be divided into upper, middle, and lower parts 1400 to 1420 of a head, as illustrated in FIG. 14. The upper part 1400 may include a brainpan, that is, an upper part of the head. The middle part 1410 may include eyes, nose, and ears parts. In addition, the lower part 1420 may include mouth, lips, cheeks, and neck parts. In this case, for example, a zone selected by the user may be changed in color like in animation or a portion around an edge of the selected zone may shine so as to be displayed like aureole.

In response to a detailed zone being selected in the screen image of FIG. 13(a), the user terminal apparatus 1 100-1 may display a label 1320 indicating various types of symptoms in a selected zone 1310, as illustrated in FIG. 13(b). The label 1320 may be a kind of sign indicating a type of symptom. For example, in FIG. 13(b), when the selected zone 1310 corresponds to the upper part 1400 of the head part of FIG. 14, a portion with symptom may be indicated in detail. Here, the label 1320 indicating a type of symptom may be displayed in the form of points or bubble with texts.

When a user moves or rotates the user model 1300 with the selected zone 1310, the label 1320 associated with symptom may disappear during the rotation and then reappear when the rotation is terminated.

In addition, when the user selects the label 1320 of specific symptom in the image of FIG. 13(b), a wizard 1330, that is, a wizard window may be displayed in a right portion of a screen image, for example, as illustrated in FIG. 13(c). In addition, in response to the corresponding wizard 1330 being pushed to the left, the wizard 1330 may disappear. Since it is impossible to display all types of symptoms in the user model 1300, the wizard 1330 may be usefully used to display a diagnosis state in detail.

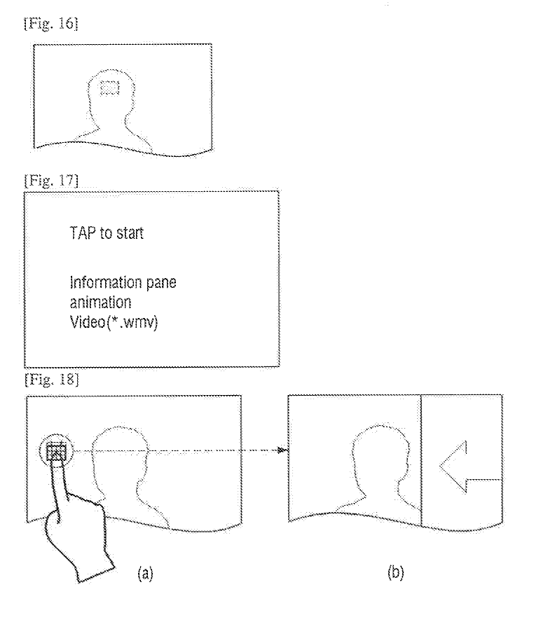

FIG. 15 is a diagram illustrating a procedure for additionally recording symptom. FIG. 16 is a diagram illustrating the case in which selected symptom is displayed in a user model when a user selects the symptom. FIG. 17 is a diagram illustrating an example of fly out animation.

An image of FIG. 15(a) may correspond to, for example, a portion (e.g., wizard) of the image of FIG. 11. For convenience of description, referring to FIGS. 15 to 17 together with FIG. 1, according to user request for selection of detailed symptom, etc. as illustrated in FIG. 11, the user terminal apparatus 1 100-1 of FIG. 1 may display an image as illustrated in FIG. 15(a). The image of FIG. 15(a) may include a predefined list associated with symptom and buttons for adding symptoms, as illustrated in FIG. 11.

When the user selects symptom of the predefined list in the image of FIG. 15(a), the user terminal apparatus 1 100-1 may display the selected symptom in a user model on a body part image positioned in a left portion of FIG. 11, as illustrated in FIG. 16. In addition, when the user selects a button NEXT, an image of FIG. 15(b) may gradually appear while the image of FIG. 15(a) disappears. The user may add voice comment or add a captured picture on the image of FIG. 15(b). Needless to say, the picture may be extemporarily captured and then added.

In addition, in response to a button PRIOR being selected in the image of FIG. 15(b), the image of FIG. 15(a) may be restored, but when the user selects the button NEXT, an image of FIG. 15(c) may gradually appear while the image of FIG. 15(b) may disappear. The user may input a detailed date through the image of FIG. 15(c). Needless to say, in response to the image of FIG. 15(c) being opened, a today date may be displayed as default. Accordingly, when the user does not determine a separate date, is may be deemed that a today date is determined as related symptom.

Then when the user selects the button NEXT in the image of FIG. 15(c), the image of FIG. 15(c), for example, a wizard may disappear and an image of FIG. 15(d) may appear. In other words, a label associated with terminated symptom of a specific part may be displayed, and the image of FIG. 15(d) may be displayed so as to allow the user to select another symptom. This may be interpreted as if the image of FIG. 10 or 11 is restored. During this process, in order to indicate termination of a wizard to the user, the user terminal apparatus 1 100-1 may temporally display an image of FIG. 17 during image conversion.

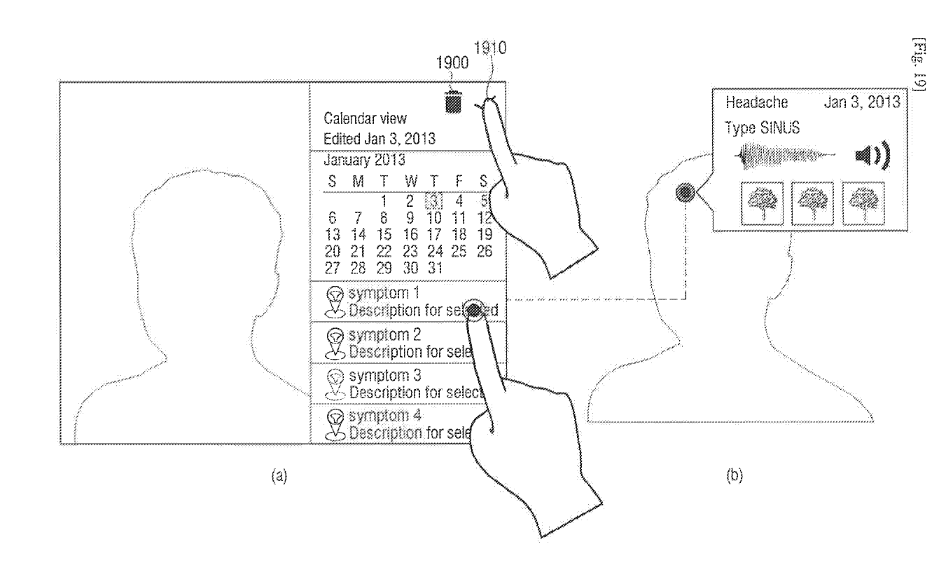

FIG. 18 is a diagram illustrating an example of an image when a calendar item of an image is selected. FIG. 19 is a diagram illustrating an example of an image of FIG. 18(b).

Images of FIGS. 18(a) and 18(b) may correspond to portions of the images of FIGS. 9 and 12. For convenience of description, referring to FIGS. 18 and 19 together with FIGS. 1 and 8, for example, when the user selects the calendar in the image of FIG. 18(a), the user terminal apparatus 1 100-1 may gradually display a calendar window from a right portion, as illustrated in FIG. 18(b).

Here, the calendar window may be embodied as illustrated in FIG. 19. In other words, a calendar may be displayed in the middle of the calendar window, and a symptom list or symptom of a specific date may be displayed in a lower portion of the calendar window. In addition, a delete icon or a restoration icon may be displayed in an upper portion of the calendar window. In response to the delete icon being selected, selected symptom may be deleted. In response to the restoration icon being selected, the calendar window may disappear. In addition, in response to a specific date being selected, a list of symptoms input at a corresponding date may be displayed in a lower portion. In response to one symptom being input at the corresponding date, the graphic image 830a may be displayed as illustrated n FIG. 12.

For example, when the user selects specific symptom of the symptom list displayed on an image of FIG. 19(a), the user terminal apparatus 1 100-1 may shown data of corresponding symptom on a user model displayed in the image of FIG. 19(a), as illustrated in FIG. 19(b). Here, the input data may refer to a graphic image, voice comment, a picture, and so on, which are selected from a predefined list. In this case, symptom information may be provided in the form of a popup window as illustrated in FIG. 19(b), but not in the form of a text label as illustrated in FIG. 13(b).

Although the image of FIG. 19(b) may be integrated with the image of FIG. 19(a), the images may be embodied as separate images, and thus exemplary embodiments of the present invention may not be particularly limited to the above image configuration.

Although all components constituting an exemplary embodiment of the present invention are integrated into one component or integrally operate, the present invention is not limited to the exemplary embodiment. That is, one or more of all the components may be selectively combined and operated within the scope of the present invention. In addition, although all the components may be embodied independent hardware devices, respectively, all or some of the components may be selectively combined to be embodied as a computer program including a program module that performs all or some functions obtained by combining one or a plurality of hardware devices. Codes and code segments constituting the computer program may be easily inferred by one of ordinary skill in the art. The computer program may be stored in a non-transitory computer readable media and read and executed by a computer and thus may embody an exemplary embodiment of the present invention.

The non-transitory computer readable medium is a medium which does not store data temporarily such as a register, cash, and memory but stores data semi-permanently and is readable by devices. More specifically, the aforementioned applications or programs may be stored in the non-transitory computer readable media such as compact disks (CDs), digital video disks (DVDs), hard disks, Blu-ray disks, universal serial buses (USBs), memory cards, and read-only memory (ROM).

The foregoing exemplary embodiments and advantages are merely exemplary and are not to be construed as limiting the present invention. The present teaching can be readily applied to other types of apparatuses. Also, the description of the exemplary embodiments of the present invention is intended to be illustrative, and not to limit the scope of the claims, and many alternatives, modifications, and variations will be apparent to those skilled in the art.

### INDUSTRIAL APPLICABILITY

### Sequence Listing Free Text

1. A user terminal apparatus comprising:
a display configured to display a user model personalized to a user and to recommend a plurality of graphic images associated with a physical condition of a user on one region of the displayed user model; and
an information visualization processor configured to, in response to one image being selected from the plurality of recommended graphic images, control the display to apply the selected graphic image to the user model.

2. The user terminal apparatus as claimed in claim 1, further comprising:
a storage configured to store medical information; and
a sensor configured to acquire data associated with the physical condition,
wherein the information visualization processor recommend the plurality of graphic images based on at least one of the stored medical information and the acquired data.

3. The user terminal apparatus as claimed in claim 2, wherein the sensor comprises at least one sensor for detection of a physical activity level of the user.

4. The user terminal apparatus as claimed in claim 2, further comprising a communication interface operatively associated with a wearable device that the user wears in order to measure the physical condition of the user,
wherein the information visualization processor acquires data associated with the physical condition through the communication interface.

5. The user terminal apparatus as claimed in claim 1, wherein the information visualization processor displays different types of symptom associated with the physical condition as the plurality of graphic images.

6. The user terminal apparatus as claimed in claim 1, wherein:
the display further displays a calendar showing the physical condition of the user according to change of data, and in response to a date being selected from the calendar, further displays a graphic image of the selected date.

7. The user terminal apparatus as claimed in claim 1, wherein the information visualization processor changes information associated with the physical condition into a language selected by the user and displays the changed information on the display in order to overcome a language issue.

8. A method for driving a user terminal apparatus, the method comprising:
displaying, by a display, a user model personalized to a user and recommending a plurality of graphic images associated with a physical condition of a user on one region of the displayed user model; and
in response to one image being selected from the plurality of recommended graphic images, controlling the display to apply the selected graphic image to the user model.

9. The method as claimed in claim 8, further comprising:
storing medical information; and
acquiring data associated with the physical condition,
wherein the controlling comprises recommending the plurality of graphic images based on at least one of the stored medical information and the acquired data.

10. The method as claimed in claim 9, wherein the acquiring of the data comprises acquiring the data using at least one sensor for detection of a physical activity level of the user.

11. The method as claimed in claim 9, further comprising being operatively associating a wearable device that the user wears in order to measure the physical condition of the user,
wherein the acquiring comprises acquiring data associated with the physical condition provided by the wearable device.

12. The method as claimed in claim 8, wherein the controlling comprises displaying different types of symptom associated with the physical condition as the plurality of graphic images.

13. The method as claimed in claim 8, wherein the displaying comprises further displaying a calendar showing the physical condition of the user according to change of data, and in response to a date being selected from the calendar, further displaying a graphic image of the selected date.

14. The method as claimed in claim 8, wherein the displaying comprises changing information associated with the physical condition into a language selected by the user and displaying the changed information on the display in order to overcome a language issue.
